(19) United States
(12) Patent Application Publication
(10) Pub. No.: US 2022/0133273 A1
(43) Pub. Date: May 5, 2022

(54) TRANSPARENT ULTRASOUND TRANSDUCERS FOR PHOTOACOUSTIC IMAGING

(71) Applicant: The Penn State Research Foundation, University Park, PA (US)

(72) Inventors: Ajay DANGI, University Park, PA (US); Sumit AGRAWAL, University Park, PA (US); Sri-Rajasekhar KOTHAPALLI, University Park, PA (US)

(21) Appl. No.: 17/429,453

(22) PCT Filed: Feb. 11, 2020
(86) PCT No.: PCT/US2020/017793

§ 371 Date: Aug. 9, 2021

Related U.S. Application Data
(60) Provisional application No. 62/803,797, filed on Feb. 11, 2019.

(51) Int. Cl. A61B 8/00

(52) U.S. Cl. CPC A61B 8/4483; A61B 8/4444; A61B 8/4416

(57) ABSTRACT

The present disclosure provides photoacoustic-imaging techniques using an optically-transparent bulk piezoelectric ultrasound transducer. A PAI system is disclosed. The PAI system has an optically translucent piezoelectric substrate, and a light source capable of providing light through the transducer to a region of interest.

### CROSS-REFERENCE TO RELATED APPLICATIONS

This application claims priority to U.S. Provisional Application No. 62/803,797, filed on Feb. 11, 2019, now pending, the disclosure of which is incorporated herein by reference.

### STATEMENT REGARDING FEDERALLY SPONSORED RESEARCH

This invention was made with government support under Grant No. EB017729 awarded by the National Institutes of Health. The Government has certain rights in the invention.

### FIELD OF THE DISCLOSURE

The present disclosure relates to imaging and medical imaging, and more particularly to photoacoustic imaging.

### BACKGROUND OF THE DISCLOSURE

Photoacoustic imaging (PAI) has emerged as one of the most promising biomedical imaging modality as it maps optical contrast of deep tissue with high spatial resolution. PAI is a hybrid imaging modality in which pulsed light excitation of the tissue causes wideband ultrasound generation due to transient thermoelastic expansion of light-absorbing molecules such as melanin and hemoglobin. Subsequently, these ultrasound waves are detected by piezoelectric transducers and photoacoustic images are formed through image reconstruction methods. In PAI, imaging depth and spatial resolution are both scalable with ultrasound and optical parameters. This has enabled development of multi-scale PAI instruments capable of imaging features as small as organelles and as big as human organs. Such instruments include photoacoustic microscopes, miniaturized endoscopes, computed tomography systems, and hand-held photoacoustic devices that adapt clinical ultrasound systems for simultaneously displaying anatomical (ultrasound) and functional/molecular (photoacoustic) contrasts of the tissue. PAI has the ability to provide optical absorption based functional and molecular contrasts of deep tissue, and is therefore well suited for biomedical imaging.

A number of PAI devices have been developed. For example, optical resolution photoacoustic microscopy can image up to a few mm depth with a sub-micron to a micron scale spatial resolution. Photoacoustic tomography systems can image a whole mouse body or human breast with a spatial resolution that is better than 200 μm.

Despite these advantages, current PAI systems suffer shortcomings which pose design challenges for high-throughput photoacoustic imaging. In current PAI systems, light is delivered to the tissue either by bending the beam around the transducer or through an opening made at the center of the transducer, using complex beam geometries that confocally align both optical excitation and acoustic reception to the same spot or field of view. This leads to bulky devices, limits possible device architectures, and constraints PAI efficacy in terms of depth, resolution and speed. For example, bending light around the transducer leads to a shadow region or a blind spot in the forward field (up to an inch) of B-mode PAI devices that adapt conventional hand-held ultrasound systems, which use two light guides flanking the transducer, for dual modality ultrasound and photoacoustic imaging of several pre-clinical and clinical applications (see FIG. 18A). To circumvent this, the devices are operated with long-working distances, necessitating thick coupling medium (water or ultrasound gel) between the device and tissue. While this works for phantom and certain in-vivo imaging applications, it severely limits real-time imaging capabilities and introduces artifacts and ultrasound attenuation, especially if any bubbles are formed during prolonged imaging periods. It is even more challenging to bend light around large aperture transducers, such as 2D arrays required for real-time 3D imaging. Similarly, multiple design challenges are faced in realizing miniaturized PAI devices for space-constrained endoscopy applications, where it is desirable that light and sound share the same beam path, particularly when integrating multimodal optical, ultrasound and photoacoustic endoscopy or microscopy technologies into one device.

Co-planar optical illumination and ultrasound reception becomes even more challenging using 2D ultrasound arrays, which may provide for high-throughput volumetric photoacoustic imaging. Currently, volumetric photoacoustic imaging is done by mechanically scanning a single element or linear array of elements, but this is slow and cannot be used for real-time volumetric imaging.

The aforementioned challenges can be overcome and novel PAI schemes can be materialized, if the transducer materials can become a part of the optical system instead of an obstruction to optics, i.e. if the ultrasound transducers can be made transparent. Although all-optical ultrasound detection technologies such as optical ring resonators and photonic cavities are naturally transparent and demonstrate high photoacoustic sensitivity, they require additional optical instruments (probe lasers and detectors). Furthermore, it is challenging to integrate ultrasound excitation methods to these all-optical ultrasound detection technologies to achieve combined ultrasound and PAI of deep tissue.

### BRIEF SUMMARY OF THE DISCLOSURE

The invention may be embodied as a photoacoustic imaging (PAI) device having an optically transparent piezoelectric transducer, and a system providing optical illumination through the transducer. A transparent piezoelectric substrate, such as lithium niobate, may be coated with a transparent conductor, such as indium tin oxide (ITO). Further, the substrate may be bonded with a transparent backing layer, made of transparent epoxy or glass, to attenuate undesirable reverberations. The transparent piezoelectric substrate with the addition of the above layers may thus function as a transparent ultrasound transducer (TUT). A system of optical illumination thorough the ultrasound transducer can use an optical fiber, a free-space laser beam, an on-chip light source (vertical-cavity SELS, laser diodes, light-emitting diodes, etc. or combinations) attached to one side of the translucent ultrasound transducer, or other light sources or combinations.

For using an optical fiber as the light source, a connecting tube may be physically connected to the substrate or the coating, and an optical fiber may be physically connected to the tube. An output-end surface of the optical fiber may be placed in contact with or very near to the substrate or the coating, as the case may be. The connecting tube may be electrically conductive. An optical source (e.g., laser diode, LED, etc.) may be used to provide light to the optical fiber, for example, at an input-end surface of the optical fiber. In some embodiments, an on-chip light source, such as a VCSEL or LED light array, or the like, can be bonded to the translucent piezoelectric substrate using a few millimeters thick translucent epoxy. An array of small lenses can be pre-mounted on an arrayed light source. In this way, an array of focused light spots can be produced on the tissue surface enabling high throughput volumetric (3D) photoacoustic microscopy.

The substrate may be coated with a material that is optically transparent and electrically conductive. The substrate may have a first primary surface and a second primary surface, and coating the substrate may be accomplished by coating the first primary surface and coating the second primary surface so that the coatings do not contact each other.

In another aspect, an optical resolution photoacoustic microscopy (OR-PAM) system based on a TUT was developed, characterized, and validated using both inanimate and biological subjects. The transducer was ITO-coated with 80% optical transmission in the visible and near-infrared optical-wavelength regions, and had a center frequency of 13 MHz with a fractional photoacoustic bandwidth of 60%. The resultant transparency of the transducer facilitated a shared pathway for both light and acoustic-wave propagation.

The present approach removed the need for additional optical components (such as acoustic-optic prisms) and large-coupling media used in conventional OR-PAM systems. Instead, the presently-disclosed OR-PAM had a much smaller, lighter imaging head—the TUT itself—with minimal acoustic coupling. Imaging experiments demonstrated an SNR of 38 dB, and a lateral and axial resolution of 8.5 and 150 μm, respectively.

The TUT-based OR-PAM approach presented here showed promising results enable the miniaturization of OR-PAM for emerging wearable and high-throughput imaging applications. Additionally, the total cost of embodiments of the present TUT system is less than 50 USD, well below that of current OR-PAM setups that require additional optical components and instruments.

### DESCRIPTION OF THE DRAWINGS

For a fuller understanding of the nature and objects of the disclosure, reference should be made to the following detailed description taken in conjunction with the accompanying drawings.

FIG. 1A is a cross-sectional view of a device according to an embodiment of the present disclosure.

FIG. 1B is a cross-sectional view of a device according to another embodiment of the present disclosure.

FIG. 1C is a cross-sectional view of a device according to another embodiment of the present disclosure.

FIG. 2A is a diagram showing an optical fiber arranged with a single element transducer.

FIG. 2B is a diagram showing a light-emitting diode arranged with a single element transducer.

FIG. 2C is a diagram showing an array of light-emitting diodes arranged with a single element transducer.

FIG. 2D is a diagram showing an array of light-emitting diodes arranged with an array of transducers (i.e., a transducer having an array of elements).

FIG. 3 is an photograph of a device according to an embodiment of the present disclosure and having an optical fiber as a light source.

FIG. 4 is a graph depicting the percentage of light transmitted (transparency %) of an Indium-Tin-Oxide (ITO) coated 250 μm thick lithium niobate crystal measured in 690 nm to 970 nm range.

FIG. 5 is a graph depicting the ultrasound pulse-echo response and the frequency transform of an ultrasound echo of a transducer of an embodiment of the present disclosure.

FIG. 6 is a graph depicting pressure output of a device according to an embodiment of the present disclosure, where the distance from the ultrasound transducer is on the longitudinal axis.

FIG. 7 is a graph depicting the photoacoustic pulse response and the frequency transform of the pulse of a transducer of an embodiment of the present disclosure.

FIG. 8 is an image of a testing apparatus that includes a translucent photoacoustic imaging device according to the present disclosure, and a standard USAF test pattern.

FIG. 9 is a graph depicting the results of a linear photoacoustic scan conducted using the testing apparatus of FIG. 8.

FIG. 10 is an image of a photoacoustic imaging target formed by marking the characters “PSU” on a light-absorbing card and an illustration of a device of the present disclosure used to image the target.

FIG. 11 is an image resulting from raster scanning the target of FIG. 10 using the disclosed device.

FIG. 12 is a diagram of a device according to an embodiment of the present disclosure where an optical spot can be raster scanned through the translucent ultrasound transducer working as a stationary window.

FIG. 13 is an image resulting from raster scanning a letter “P” on a light absorbing card using a device as depicted in FIG. 12 where a 1 cm×1 cm transparent ultrasound transducer is used as the transparent window. Maximum amplitude projection photoacoustic image obtained from optical-only raster scanning over a letter ‘P’ marked on a black light absorbing card.

FIG. 14 illustrates a wearable form of the technology described herein where the transparent transducer can be vertically integrated with an array of light sources in a compact package and used for capturing human biometrics. Biometrics may be captured by, for example, co-registering ultrasound and photoacoustic image of fingerprints.

FIG. 15 illustrates a use of the technology for studying a rodent brain by monitoring its hemodynamic activity through vasculature imaging.

FIG. 16 illustrates a use of the technology incorporated into a smartphone for capturing biometrics to, for example, authenticate a user.

FIG. 17 illustrates a use of the technology described herein where the transparent ultrasound transducer can be used as a wearable patch and an optical beam can be raster scanned over the transparent window to generate a photoacoustic image.

FIG. 18A illustrates a prior art hand-held ultrasound imaging device that is adapted for combined ultrasound and photoacoustic imaging by integrating optical fibers to the device.

FIG. 18B describes a use of the transparent ultrasound technology described herein where the device can be made more efficient (e.g., without dark fields or shadows) and compact (not taking additional space for light) by providing the optical illumination through the transducer, for combined ultrasound and photoacoustic imaging.

FIG. 19 is a photograph of an optically transparent planar (10 mm×10 mm) lithium niobate substrate coated with ITO.

FIG. 20 is a block diagram representing a raster scan setup used for photoacoustic imaging using the transparent ultrasound transducer.

FIG. 21 is a graph of a photoacoustic pulse response of a transparent ultrasound transducer showing ˜6 mV amplitude from a light absorbing target. The frequency response of the photoacoustic pulse shows 14.5 MHz center frequency with ˜70% bandwidth.

FIG. 22 is a photograph showing photoacoustic imaging using the fiber-TUT device, wherein a linear photoacoustic scan was performed along a 6 cm long line covering line pairs of elements 2 to 6 in Group 2 of a USAF imaging resolution target.

FIG. 23 is a plot of peak photoacoustic amplitude along the linear scan with two points, P1 and P2, marked to estimate the resolution.

FIG. 24 is a schematic cross-sectional view of a TUT test embodiment, which had 10 mm height and 9×9 mm² field of view.

FIG. 25 is a photograph of a transducer of the test device of FIG. 24, clearly showing Nittany Lion mascot underneath.

FIG. 26 is a diagram of an optical-resolution photoacoustic-microscopy (OR-PAM) setup. Raster scanning achieved by Motor 1 moving Mirror 1 to perform x-axis scan. Motor 2 moved Mirror 2 and L3 to perform y-axis scan. BS: beam sampler; NDF: neutral density filter; PD: photodiode; PH: pinhole; L1, L2, L3: planoconvex lenses with 50, 75, and 50 mm focal lengths, respectively.

FIG. 27 are results of characterizing a test transparent lithium niobate ultrasound-transducer-based OR-PAM system. (a) Measured electrical impedance results; (b) Pulse-echo response; (c) Photoacoustic pulse response of USAF resolution test target; (d) Maximum-amplitude-projection (MAP) image of target via edge scanning. PA: photoacoustic; (e) Edge-response data and fitted line-spread-function (LSF) curve showed 8.5 μm lateral resolution. ESF: edge spread function; (f) Gaussian enveloped curve fitted profile showed 150 μm axial resolution.

FIG. 28 is a MAP image of carbon-fiber phantom with 0.5×0.5 mm² area. PA: photoacoustic.

FIG. 29A is a photograph of chick-embryo chorioallantoic membrane (CAM) with imaging area marked by white box.

FIG. 29B is a MAP image of CAM vasculature inside white box of 29A.

FIG. 30 is a photograph of a melanoma phantom.

FIG. 31A is a MAP image of melanin particles detected under mouse skin.

FIG. 31B is a color-coded depth profiling of melanoma phantom. Color bar represents depth relative to skin surface.

FIGS. 32(a) to 32(d) illustrate stages of manufacturing a transparent ultrasound transducer integrated with an optical fiber that is in keeping with embodiments of the disclosure: (a) ITO coated lithium niobate crystal mounted on a glass slide; (b) conductive bonding to brass tubing using silver epoxy (E-solder 5022); (c) filling of the backing layer after fiber insertion and center alignment; and (d) making front- and back-side electrode contacts with a coaxial wire.

### DETAILED DESCRIPTION OF THE DISCLOSURE

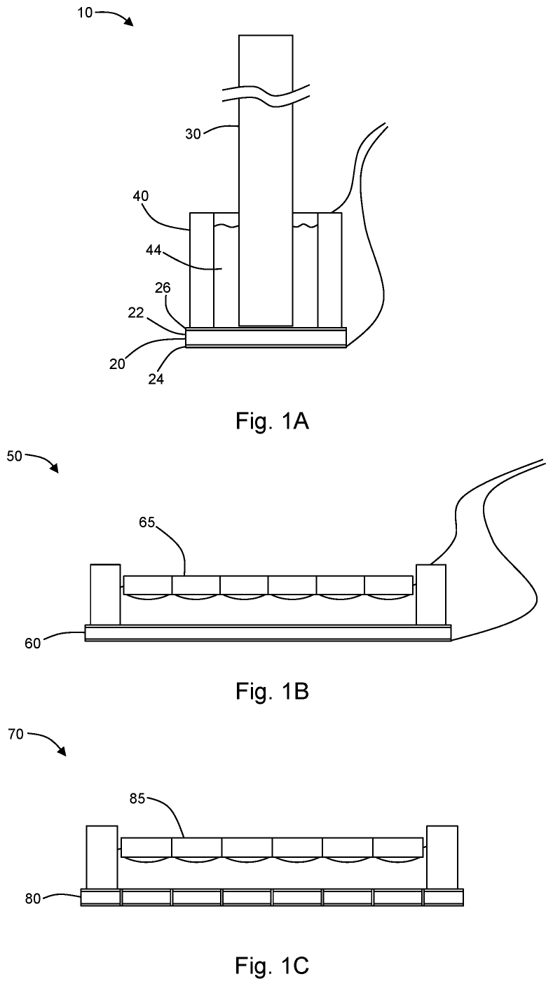

The present disclosure provides photoacoustic-imaging techniques using an optically-transparent bulk piezoelectric ultrasound transducer. With reference to FIG. 1A, the present disclosure may be embodied as a device 10 for photoacoustic imaging of a region of interest. The device 10 includes a transparent piezoelectric transducer 20 for receiving ultrasonic emissions from the region of interest. A light source 30 is configured to illuminate the region of interest by transmission through the transducer 20. It should be noted that the terms “transparent” and “translucent” are used interchangeably herein to indicate a material which can transmit all or some of a wavelength or range of wavelengths of light. The term “light” is used herein to include any electromagnetic radiation, including but not limited to visible light, ultraviolet, infrared, x-ray, and/or microwave (or portions of such radiation).

The transducer 20 has a piezoelectric substrate 22. The substrate 22 can be made from any transparent material with piezoelectric properties. For example, the substrate 22 may be made from lithium niobate (LiNbO₃), polyvinylidene fluoride (PVDF), lead magnesium niobate-lead titanate (PMN-PT), piezoelectric composites (e.g., 1-3 composites, etc.) or any other such material or combinations thereof. Lithium Niobate is a versatile optical material used in various photonic applications. A wafer of lithium niobate having polished surfaces shows an optical transparency of more than 80%. 36° Y-cut lithium niobate exhibits good electromechanical coupling coefficient (49%), high acoustic wave velocity (7340 m/s), and low permittivity (39), making it an suitable candidate for use in ultrasound transducers, particularly in receive mode. In crystalline form, LiNbO₃ has a high curie temperature (>1100° C.), which makes it well suited for PAI systems operating at high temperatures. Furthermore, the high acoustic wave velocity is beneficial for use in very high frequency (e.g., >50 MHz) bulk ultrasound transducers. Lithium niobate has also been used for high temperature ultrasound transducers due to its high Curie temperature. PVDF is another transparent piezoelectric material with the added benefit of being more flexible than ceramic material, but it exhibits much lower sensitivity due to lower electromechanical coupling. The transducer may have a thickness suitable for use with ultrasonic waves (responsive to ultrasonic frequencies). For example, as further detailed below in the example embodiments, a 250 μm lithium niobate substrate has a center frequency of approximately 15 MHz. Thinner wafers will be responsive to higher frequencies (e.g., lithium niobate with a thickness of 50 μm or less may be responsive to frequencies of 100-200 MHz). The use of higher frequencies may increase bandwidth and improve spatial resolution. On the other hand, thicker wafers will allow for imaging deeper into the region of interest.

The transducer 20 may further comprise a front electrode 24 in communication with a front surface (front side) of the substrate 22. The term “front” is used herein to indicate a component closest to the subject being imaged, and “back” is used to indicate a component opposite the subject being imaged. The front electrode 24 may be a layer of a transparent conductor on at least a portion of the front surface of the substrate. The transparent conductor may be, for example, indium-tin-oxide (ITO), graphene, a silver nanowire composite (having sparsely spread silver nanowires), a carbon fiber composite, or any other transparent conductive material or combinations of materials. ITO also can be used as an anti-reflective coating on LiNbO₃, thus improving the transparency of the piezoelectric substrate material further. The transducer 20 may further comprise a back electrode 26 in communication with a back surface (back side) of the substrate 22. The back electrode 26 may be a layer of a transparent conductor on at least a portion of the back surface of the substrate, such as, for example, ITO, or other such materials or combinations of materials.

Transducers of the present disclosure may be of various sizes. For example, transducers having diameters of less than 2.5 mm, 1 mm, or less, may be suitable for uses such as endoscopy. Embodiments using a TUT for OR-PAM may utilize larger sizes such as 5 mm×5 mm, 10 mm×10 mm, or larger or other sizes between these values. The sizes described herein are intended to be exemplary, and transducers are not limited to these sizes. Transducers may be larger, smaller, or sizes in between the values disclosed here. Transducers may also take on various shapes. For example, a transducer may be round (circular, ovoid, etc.), rectilinear (square, rectangular, etc.), or any other regular or irregular shape as suited for a particular application.

The light source of the device 10 may be an optical fiber 30. Such an optical fiber may receive light at an input end and emit the received light at an output end. For example, the optical fiber 30 may be configured to be coupled to a laser at an input end and to emit light received from the laser at an output end opposite the coupled end. In some embodiments, an output end of the optical fiber is attached to the back side of the transducer and positioned such that light emitted from the output end passes through the transducer to illuminate the region of interest. In some embodiments, the light source comprises one or more lasers, such as, for example, a vertical-cavity surface-emitting-laser (VCSEL). In some embodiments, the light source comprises one or more light-emitting diode (LEDs). Other light sources are known and may be used in the device. Combinations of two or more types of light sources may be used. The light source may emit light having a source wavelength range. For example, the source wavelength range may be 250 nm-2400 nm. In another example, the source wavelength range may be 690-970 nm. Other appropriate ranges will be apparent in light of the present disclosure. A suitable transducer is transparent in the source wavelength range. For example, a suitable transducer may allow at least 30% of light in the source wavelength range to be pass through. In some embodiments, suitable transducers may allow at least 50%, 60%, 70%, 80%, or 90% of light in the source wavelength range to pass through (i.e., transparent/translucent).

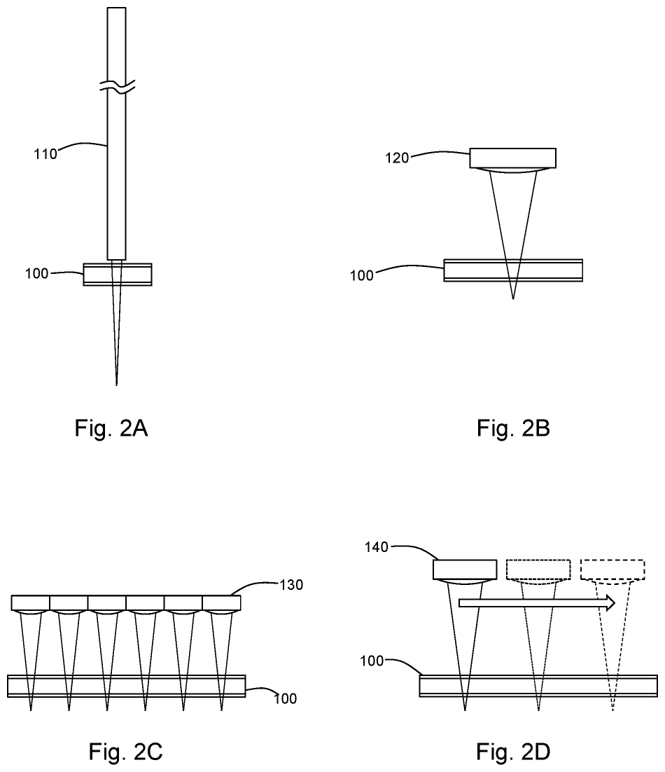

Illumination through the transducer can be achieved by various systems depending on the application. FIGS. 2A-2D are simplified diagrams showing example arrangements of light sources with respect to a transducer. For example, for space-constrained applications, such as, for example, endoscopy, etc., a single-element transducer 100 can be integrated with an optical fiber 110 (see, e.g., FIG. 2A). FIG. 2B shows a similar embodiment using an LED light source 120. In another arrangement, an arrayed light source 130 (for example, an array of LEDs or the like) can be arranged to illuminate a region of interest through a transducer 100 (see, e.g., FIG. 2C). In some embodiments, each element of the light source array (or combinations of more than one element) may be activated separately, sequentially, and/or simultaneously, according to the application.

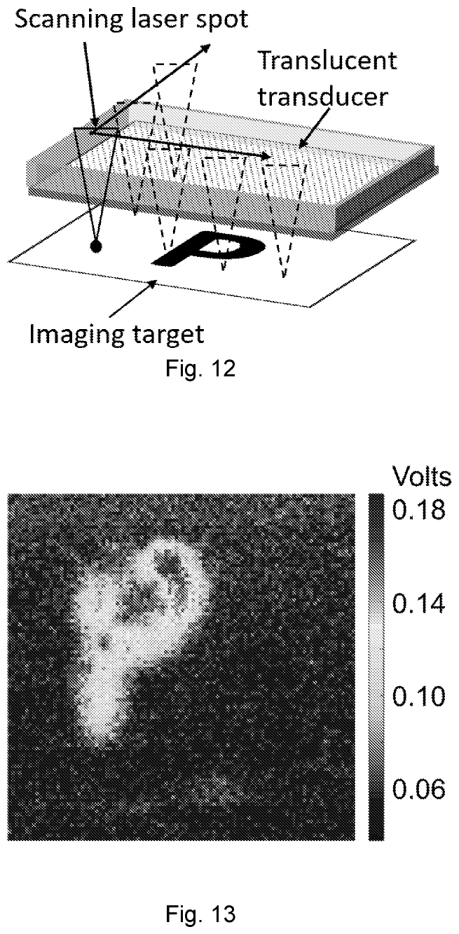

In another embodiment, sometimes referred to as a “window transducer” embodiment, a light source 140 (which may be a single-element light source or a multi-element light source) can be moveable with respect to the transducer 100 such that the light source 140 can be scanned (e.g., raster scanned) over the transducer to image a region of interest without need to move the transducer. By doing so, unlike in the prior art photoacoustic systems, the optical hardware may be separated from the ultrasound acquisition. This may be advantageous in imaging conditions where other imaging methods need to be applied on the same location. For example, such an embodiment can be used for simultaneous photoacoustic and optical imaging of a region of interest. FIG. 12 describes a system of illumination and imaging for such a transducer. Such embodiments can be advantageous for high-throughput PAI.

The device 10 may comprise a housing 40. The housing 40 may provide structural support for the transducer 20 and/or other components. The housing may be made from any material or combinations of materials suitable for the application. In some embodiments, the housing is made from a metal, such as, for example, brass. In this way, the housing may be arranged to be in contact with an electrode of the transducer and provide a convenient way to connect to the electrode. The housing may also provide an acoustic dampening function by, for example, absorbing reverberations. In this way, bandwidth and sensitivity of the device may be improved.

The device 10 may further comprise a transparent backing layer 44 disposed on the back side of the transducer 20. Such a backing layer 44 may provide dampening to improve bandwidth and sensitivity. The backing layer may be, for example, a transparent epoxy, or any other transparent material or combinations of materials. The bandwidth of a device may be further improved by adding particles, such as tungsten or silver particles, to the backing layer to increase the mass of the dampener. A device may advantageously avoid disposing such particles between the light source and the transducer. Mechanically coupling the light source with the transducer can lead to improved acoustic dampening of acoustic waves received by the substrate.

FIG. 1B depicts an embodiment of a device 50 having a single-element transducer 60 and an arrayed light source 65. In such an embodiment, one or more elements of the arrayed light source may be energized at the same time and the acoustic response may be detected by the transducer. Then, a different one or more of the elements of the arrayed light source may be energized at the same time and another acoustic response detected. FIG. 1C depicts an embodiment of a device 70 having a multi-element (e.g., arrayed) transducer 80 and an arrayed light source 85. In this embodiment, one or more elements of the arrayed light source may be energized at the same time and the acoustic response may be detected by one or more elements of the arrayed transducer. In a particular example, a single element of the light source may be energized, and one or more elements of the transducer may be used to detect the acoustic response.

In some embodiments, the device may be used for hybrid ultrasound/photoacoustic imaging. In such a device, the transducer may be actuated to provide an excitation signal (e.g., pulse, pulse train, etc.) to the region of interest. The device may include a detector such as an image sensor (e.g., charge-coupled device (CCD), CMOS sensor, etc.) to monitor the region of interest for changes. In some embodiments, the transducer may also be used to receive a resulting ultrasonic emission from the region of interest. In some embodiments, the transducer may include one or more elements for providing ultrasonic excitation to the region of interest and one or more elements for receiving a resulting ultrasonic emission from the region of interest.

Photoacoustic imaging systems manufactured in keeping with the present disclosure may be used for non-invasively imaging light or other electromagnetic absorptions inside a tissue or other material, and distinguishing key absorbents based on their characteristic absorption spectrum over a broad wavelength electromagnetic radiation. For example, the technology may be useful for imaging oxy and de-oxyhemoglobin to map vascular networks of arteries and veins and monitoring hemodynamic activity inside the body.

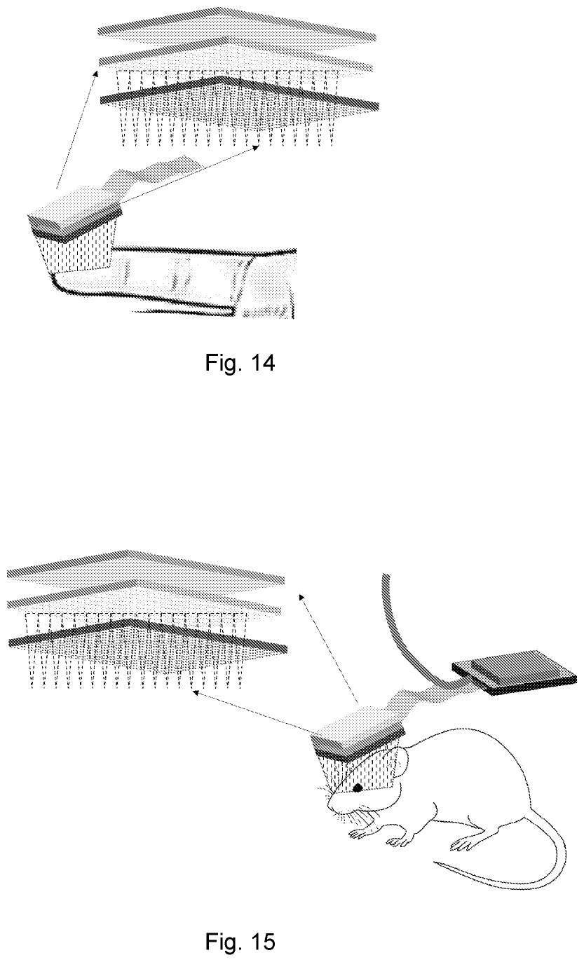

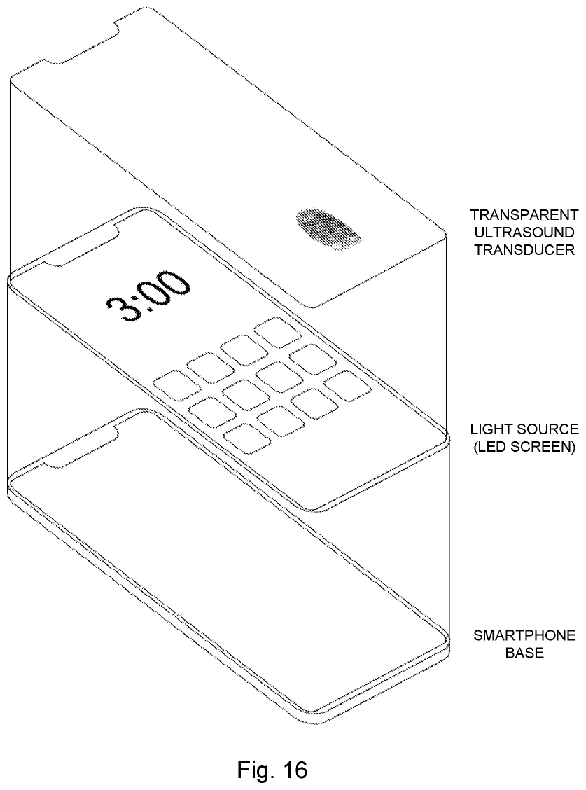

The through transducer illumination system may be used for various photoacoustic imaging applications in different embodiments. For example, a transparent ultrasound transducer can be vertically integrated with an arrayed light source to form a single chip solution for photoacoustic imaging which can be compact or wearable. In an example of a wearable form, a device can be used for biometric sensing applications such as fingerprint capture, for example as illustrated in FIG. 14. Such applications may also use a hybrid ultrasound/photoacoustic imaging approach. In another example, a device can be mounted on the head of a rodent as illustrated in FIG. 15 for frequent monitoring or multi-modal imaging useful in neurovascular research. In another example, a device may be integrated into a system such as a computer, a tablet, a smartphone, etc. to capture biometric information. FIG. 16 shows an example of a smartphone having a transparent ultrasound transducer layer backed by an LED screen as a light source. Such a solution may be used to capture a biometric for user authentication or other functions. In some embodiments, a transparent ultrasound transducer may be operated (e.g., actuated) to excite a region of interest and a detector may be used to monitor the region of interest for changes. For example, in the smartphone embodiment, the transparent ultrasound transducer may be used to excite a fingertip and a cell phone camera or other detector may be used to monitor the fingertip for a response based on the ultrasound excitation.

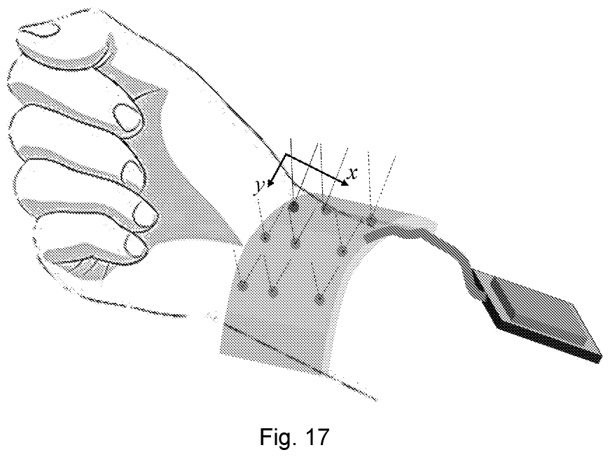

The transducer system shown in FIG. 12 can be particularly useful as a wearable transparent patch that can be placed on a body part. In this way bulky optical systems can be separated from the imaging hardware and one or many focused optical spots can be raster scanned through the transducer to generate photoacoustic images. An example of this embodiment of the invention is illustrated in FIG. 17.

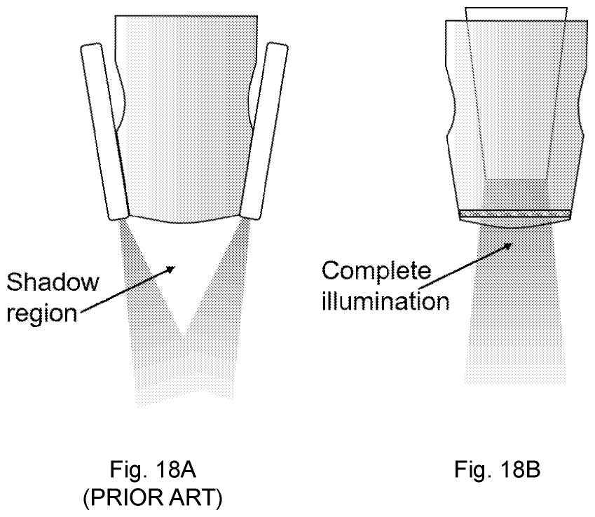

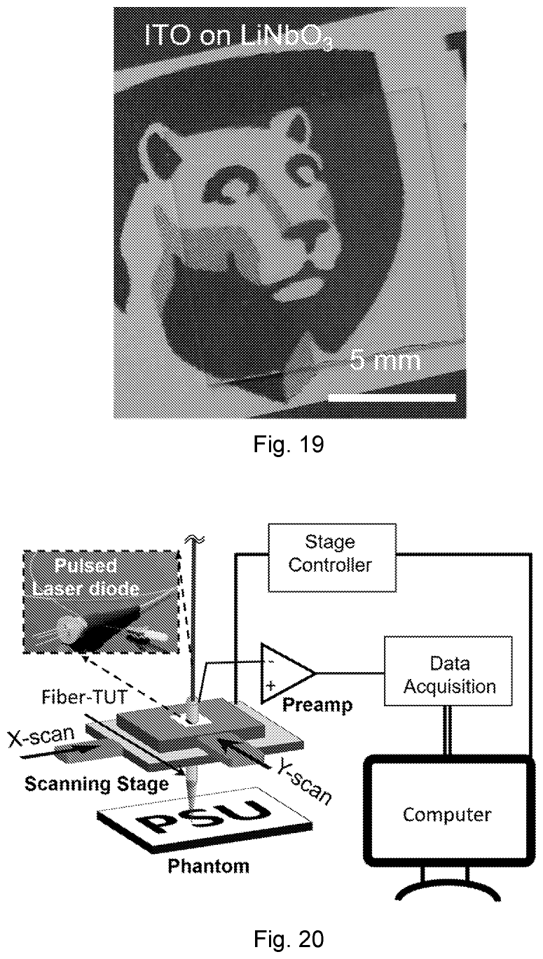

A transparent ultrasound transducer comprising a linear (1D) or 2D array of elements may be packaged with optical fibers illuminating light through the transducer (for example, as illustrated in FIG. 18B), for combined/co-registered ultrasound and photoacoustic imaging. Such a co-registered ultrasound and photoacoustic imaging device may be more compact and capable of providing better field of view without any (or a very small) dark region near the surface of the device. The presently-disclosed device can be compatible with convention clinical ultrasound electronics.

Embodiments of the present disclosure are relatively low-cost, easy to manufacture, compatible with commonly used clinical ultrasound electronics, and scalable for different configurations including two-dimensional arrays to achieve real-time three-dimensional photoacoustic imaging. The use of transparent (i.e., translucent) ultrasound transducers allows the transducer to be part of optical system instead of an obstruction to the optics. By doing so, the presently-disclosed device may be more compact and portable than prior-art PAI systems.

In another aspect, the present disclosure may be embodied as a method for photoacoustic imaging a region of interest. In such a method, a transparent piezoelectric transducer is provided. A first portion of the region of interest is illuminated through the transducer. The first portion may comprise the entire region of interest or a part of the region of interest. In an example, a light source may be configured to illuminate the first portion of the region of interest by transmitting light through the transparent transducer. The method includes receiving an ultrasonic emission from the first portion of the region of interest, wherein the ultrasonic emission results from the illumination.

The method may further include moving the transducer to a second portion of the region of interest. The second portion of the region of interest is illuminated through the transducer. The method includes receiving an ultrasonic emission from the second portion of the region of interest, wherein the ultrasonic emission results from the illumination. The steps are repeated for additional portions of the region of interest. In this way, the entire region of interest may be imaged. In some embodiments, the second portion of the region of interest is illuminated and a resulting ultrasonic emission is received without moving the transducer. For example, the transducer may be of a size sufficient to image the region of interest without moving the transducer. In such embodiments, a light source may be raster scanned in order to illuminate the second (and additional) portions. In some embodiments, the light source is an array of elements and may be used to illuminate the second (and additional) portions.

In some embodiments, the method may include actuating the transducer to excite at least a portion of the region of interest. It is known that piezoelectric materials can be used as sensors (for example, by detecting a voltage across the material) and/or as actuators (for example, by applying a voltage across the material). In some embodiments, the transducer may be used for hybrid ultrasound/photoacoustic imaging by actuating the transducer to excite at least a portion of the region of interest and receiving an ultrasonic emission resulting from the ultrasonic excitation. In some embodiments, the method may include monitoring the region of interest for changes using a detector, such as an optical detector (e.g., charge-coupled device (CCD), CMOS sensor, etc.)

### EXPERIMENTAL EMBODIMENTS

Experimental (test) embodiments are described throughout the disclosure solely to illustrate embodiments of the disclosed device and are not intending to be limiting.

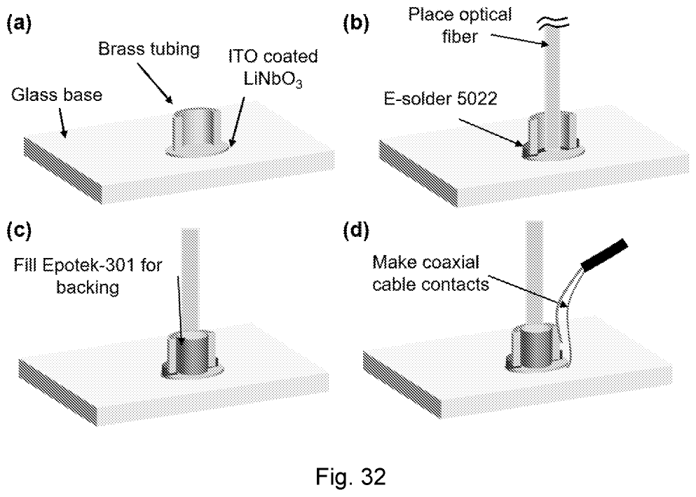

An experimental embodiment was fabricated using a transducer made from a 36° Y-cut LiNbO₃ substrate with a thickness of 250 μm. The LiNbO₃ substrate was coated with a 200 nm thick ITO on both sides by sputtering at 300° C. in a 15 milli-torr argon environment using 200 watts forward power (FIG. 2b). The ITO-coated LiNbO₃ substrate was divided into appropriately sized pieces for two test embodiments. In a first test embodiment, the substrate was cut into a 2.5 mm×2.5 mm piece. The 2.5 mm×2.5 mm piece was mounted on a glass base using wax (FIG. 32a). A housing was made from a brass tube with a 2.3 mm outer diameter. The brass tube was partially dipped in a conductive silver epoxy (E-solder 3022, Von Roll Isola Inc., CT, USA) at one end, and then pressed against the back side of the LiNbO₃ piece and cured to achieve a strong conductive bond between the ITO coated LiNbO₃ and the tube (FIG. 32b). A polished optical fiber with a 400 μm diameter core was aligned with the center of the brass tube, and placed against the back side of the LiNbO₃ piece (FIG. 32b). While maintaining this assembly, a non-conducting transparent epoxy (Epotek-301, Epoxy Technologies Inc., MA, USA) was poured into the brass tubing, which served the purposes of ultrasound backing (dampening) and also ensured a solid fiber-transducer assembly with no particles or spacing between the front surface of the optical fiber and the backside of the LiNbO₃ piece (FIG. 32c). The shield wire of a micro-coaxial cable was soldered to the brass tube to electrically access the backside ITO electrode and the core of the micro-coaxial cable was soldered to the top ITO electrode (FIG. 32d). The opposite end (input end) of the optical fiber was coupled to a 905 nm pulsed laser diode (PLD) (905D2S3J09, Laser Components Inc., NH, USA) which delivered 65 W peak optical power at the output end of the fiber. The PLD was driven using 50 ns pulsed voltage input at 20 kHz repetition rate.

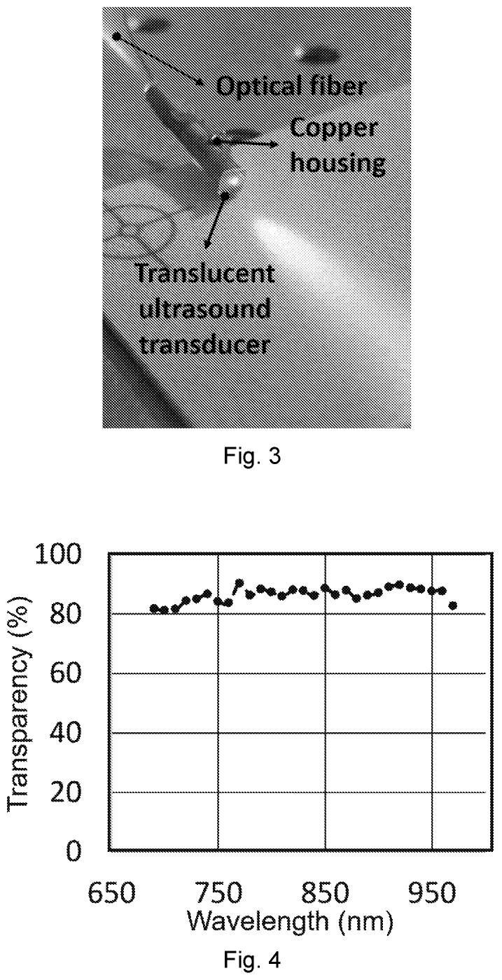

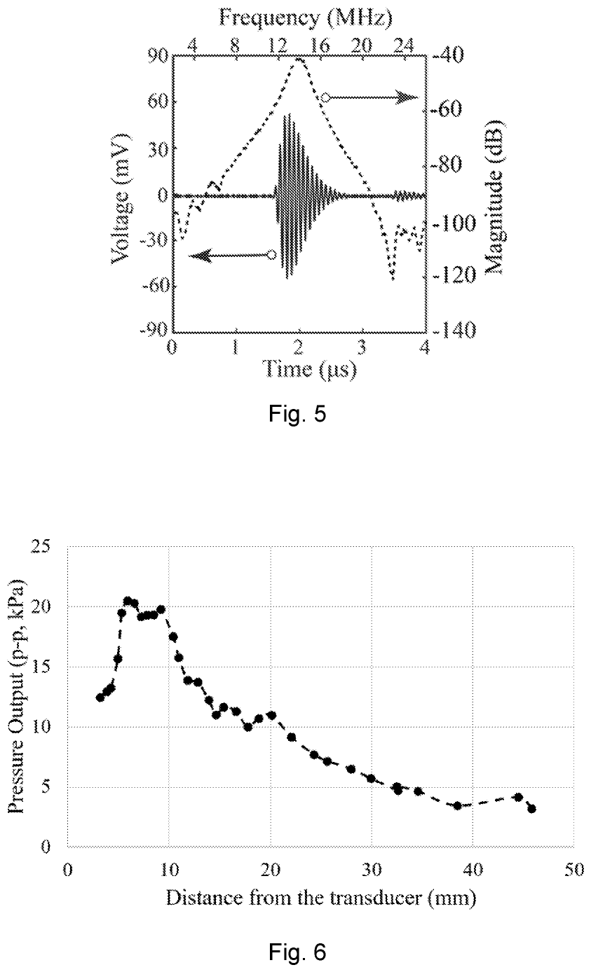

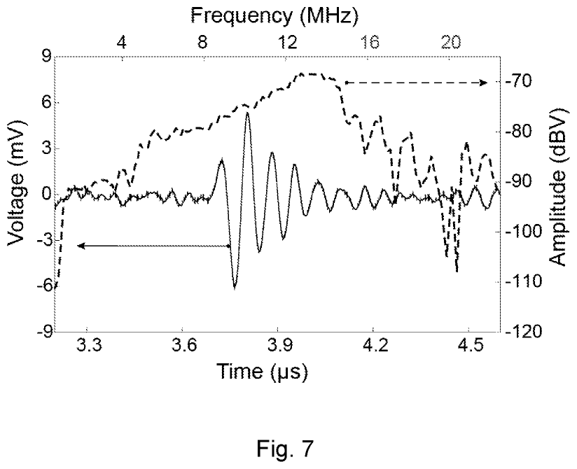

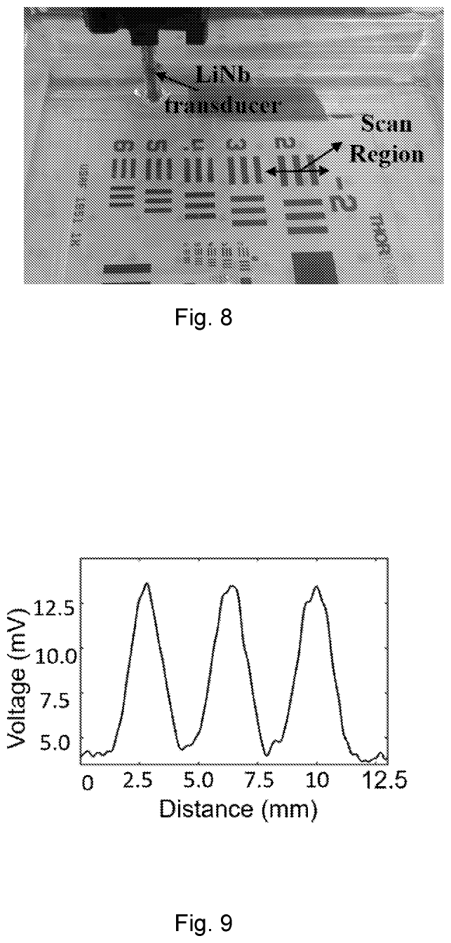

FIG. 3 shows the light output through the 2.5 mm square piece of LiNbO₃ of the test embodiment and irradiating an infrared detection card. The optical transparency of the resulting device was evaluated. The optical transparency of the fiber-coupled 2.5 mm TUT in the wavelength range of 690 nm to 970 nm was estimated by measuring the reduction in the light output with and without the LiNbO₃ between a tunable light source and a photodetector (Det10A, Thorlabs Inc., NJ, USA). A 1 mm diameter optical fiber bundle connected to a tunable laser (Phocus Mobile, Opotek Inc., CA, USA) was used as the tunable light source for these measurements. As shown in FIG. 4, the transducer demonstrated >80% transparency over this wavelength range. The test device was also characterized for its ultrasound pulse-echo response. The transducer was kept at ˜3 cm axial distance from a flat aluminum block and excited using a 180 V pulse having ˜20 ns pulse-width generated by a standard ultrasound pulse-echo amplifier (Olympus 5073PR, Olympus NDT Inc., MA, USA). The reflected echo measured a ˜120 mV peak-to-peak amplitude for 0 dB receiver gain and its frequency response showed a center frequency of 14.5 MHz with ˜30% bandwidth (FIG. 5). The ultrasound pressure output of the test device was measured by actuating the transducer using a 10 cycle sinusoidal burst of 5 Vpp input at 14.5 MHz frequency, while a commercial hydrophone (HGL-085, Onda Corp., CA, USA) was scanned along the longitudinal axis of the transducer. The hydrophone output captured from 3 mm to 46 mm axial distance shows a peak pressure of ˜20 kPa at ˜6 mm distance from the transducer, beyond which the characteristic far-field pressure reduction inversely proportional to the distance was observed.

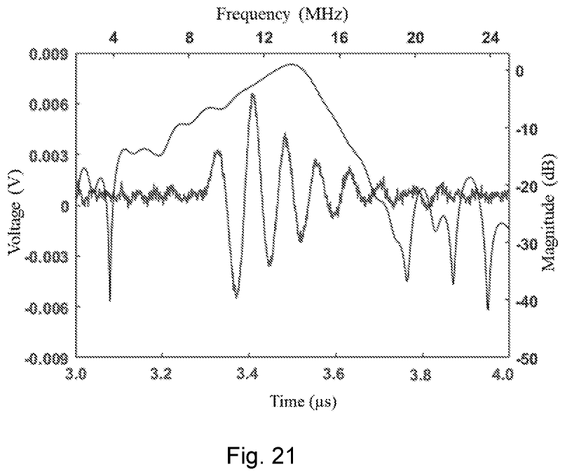

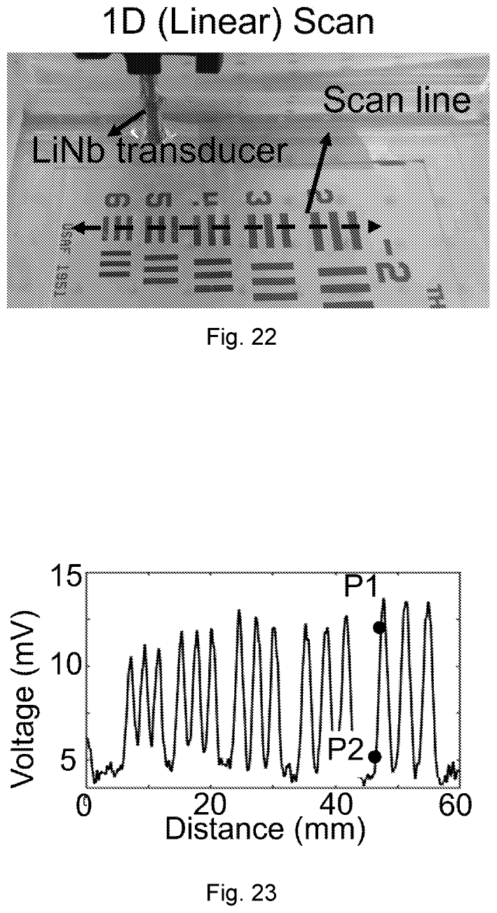

In addition, the photoacoustic response was measured. To characterize the photoacoustic response of the device, a light absorbing black card was placed at ˜5 mm distance from the transducer surface in underwater condition. The output signal from the ultrasound transducer was fed to a preamplifier (Olympus 5073PR, Olympus NDT Inc., MA, USA) providing 39 dB gain, and then digitized using a high speed (1 GSps) 16-bit data acquisition system (Razormax-16, Dynamic Signals LLC, IL, USA). FIG. 20 shows a schematic representation of the experimental photoacoustic imaging system. The received photoacoustic signal clearly showed a single sharp pulse at ˜3.3 μs, corresponding to the location of the card, (FIG. 21), with ˜12 mV peak-to-peak amplitude. The frequency domain transform of the photoacoustic pulse response showed ˜70% fractional bandwidth. The resolution capabilities of the fiber-TUT device were tested by scanning the line pairs of Group-2, Elements 2 to 6 of a standard USAF resolution target (R3L3S1P, Thorlabs Inc., NJ, USA) and plotting the peak amplitude of the photoacoustic response generated by the light-absorbing gold-coated lines (FIGS. 22 and 23). The edge spread function of one of the scanned line pairs showed a spatial resolution of ˜900 μm, as estimated for a drop from 90% amplitude (P1) to 10% amplitude (P2) across the edge. The resolution was low due to the unfocused nature of the light output from the multimode optical fiber in the experimental device, which resulted in diffused optical illumination on the target. The imaging resolution can be improved using techniques such as, for example, fiber coupled graded-index (GRIN) lenses for optical resolution photoacoustic microscopy or adding an acoustic lens on the top surface of the LiNbO₃ surface for acoustic resolution photoacoustic microscopy.

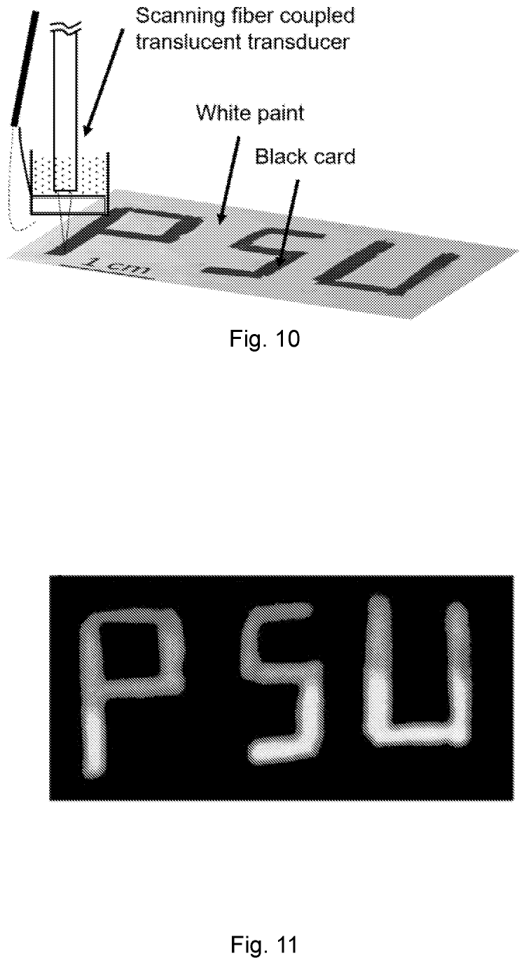

The experimental fiber-TUT device was mounted on a 3-axis stage (NRT-1000, Thorlabs Inc., NJ, USA) and raster scanned over the letters “PSU” marked on a light-absorbing black card, as shown in FIG. 10, with a 100 μm step size covering a 20 mm×35 mm region. The photoacoustic signal received at each point was post-processed using an image reconstruction program written in Matlab (Mathworks Inc., MA, USA) to form the maximum amplitude projection (MAP) photoacoustic image reading “PSU” shown in FIG. 11.

### Window Transducer Test Embodiment

In a second embodiment, a 10×10 mm transducer was used with a scanned light source. For fabricating the 10 mm×10 mm window TUT device, a 1 mm high square brass ring was used for casing and backside epoxy filling, in order to minimize aberrations in the light passing through. The window transducer was mounted above a letter ‘P’ marked in white on a black card. An optical fiber was raster scanned across the window TUT by holding the fiber approximately 5 mm above the TUT in air, while the TUT was water-coupled to the phantom black card (FIG. 12). FIG. 13 shows the MAP photoacoustic image of the target “P” reconstructed from 3D volumetric photoacoustic data.

The non-uniform intensity in the reconstructed image was most likely due to some variations in the light-absorbing regions of the target. Despite this limitation, the imaging result shows the feasibility of optical-only scanning based PAI enabled by the use of a window TUT. Although the study was limited to the single element TUTs, fabrication of 1D and 2D TUT arrays that allow light delivery through the transducer using free space coupling or arrayed light sources (e.g., optical fiber bundles, etc.) are within the scope of the present disclosure and may be useful for real time PAI applications. Such PAI devices can be directly operated with conventional ultrasound systems as long as the operating frequency is within the sampling frequency of the data acquisition system.

### Optical-Resolution Photoacoustic Microscopy (OR-PAM) Embodiment

Optical-resolution photoacoustic microscopy (OR-PAM) has recently gained significant attention from the biomedical-imaging community as it provides label-free optical contrast from physiologically relevant tissue chromophores that are located a few millimeters deep, with subcellular spatial resolution. In OR-PAM, a tightly focused laser pulse illuminates the tissue and generates wideband acoustic waves from light-absorbing chromophores that are then detected by an acoustic transducer. Time-resolved photoacoustic waves, in combination with the two-dimensional raster scanning along the x-y plane (lateral dimension), generate three-dimensional data from which maximum amplitude projection (MAP) and volume-rendered photoacoustic images can be created.

Conventional OR-PAM setups use complex imaging geometries to coaxially align optical illumination and acoustic detection paths. In early OR-PAM setups, coaxial alignment was achieved using an acoustic-optic prism combiner consisting of one right-angle prism and one rhomboid prism pressed tightly to a thin layer of silicone oil. The laser light was focused by a system of optical lenses and then passed through the prism combiner before irradiating the tissue. A correction lens was attached to the prism combiner to refocus the light that was defocused through the combiner. Tissue-generated photoacoustic waves propagated through the rhomboid prism and were reflected by the silicone oil layer into the ultrasound detector attached to the prism. Since the entire imaging head, consisting of the above acoustic-optic prism combiner, the transducer, and the focused light, was moved to scan the subject, these systems exhibited slow acquisition speed, limited field of view (FOV), and significant acoustic loss.

The current generation of OR-PAMs reflect the light, instead of the acoustic waves, by sandwiching an aluminum foil in the acoustic-optic combiner. This allows dual axis optic only scanning using a two dimensional galvo mirror to improve the image acquisition speed and generate a wide FOV. The entire imaging head, including the galvo mirror, is submerged in a large volume (70×40×20 mm³) of a nonconducting liquid coupling medium that rests above the imaged subject. Such a bulky imaging head limits high throughput and wearable imaging applications because it constrains animal imaging performed under anesthesia and causes discomfort to living subjects. Moreover, acoustic loss here is still significant because acoustic waves travel through the large coupling medium and the prism combiner before being detected by the transducer.

Alternatively, some OR-PAMs include a ring-shaped single-element ultrasound transducer to eliminate the off-axis alignment problems of optical illumination and acoustic detection. The focused light is directly delivered through a hole at the center of the transducer, or coupled using a single-mode fiber integrated with a gradient-index (GRIN) lens. The imaging head is then two-dimensionally raster-scanned using mechanical stages to generate volumetric images. Although the imaging head is miniaturized in these OR-PAM systems, the FOV, numerical aperture, and imaging speed (due to physical scanning of the imaging head) are still limited. Besides, they still require a-few-millimeter thick water coupling medium above the imaged subject due to long working distances.

The above drawbacks of conventional OR-PAM systems can be addressed if ultrasound detectors are transparent to light. To achieve this, all-optical ultrasound detection technologies, such as Fabry-Pérot etalons, microring resonators, and other photonic integrated circuits were studied for PAM. Although these are transparent technologies offering high photoacoustic sensitivity, they require complex fiber integration with an additional laser source and other optical-detection instruments. More importantly, they lack ultrasound excitation capabilities for applications that require combined ultrasound sensing and ultrasound tissue stimulation. Recently, transparent capacitive micromachined ultrasonic transducers (CMUTs) were developed. However, CMUTs need specialized front-end application-specific integrated circuits (ASICs), and involve a complicated fabrication process inside a cleanroom.

The present disclosure provides a device for OR-PAM using a TUT. Extending the window-transducer approach described above, the present-disclosed OR-PAM technique allows the optical-only scanning of a tightly focused light beam through a transparent-ultrasound-transducer (TUT) window. Such a device may be used to image biological samples. This TUT-based OR-PAM approach simplifies the coaxial alignment of optic and acoustic paths without the need for additional optical components (such as acoustic-optic prism combiners and correction lenses) and a large acoustic-coupling medium. The present approach allows for a TUT to be fixed onto an imaging object (such as the skull of a mouse) to facilitate wearable imaging without a thick coupling medium. This can enable applications such as imaging the brains of freely behaving or awake mice in combination with ultrasound stimulation. Additionally, depending on the size of the TUT, this approach can enable high-speed scanning of large areas with single-channel data acquisition.

### OR-PAM Test Embodiment

An experimental OR-PAM system was built and characterized. The experimental system is described here to illustrate an embodiment of the present disclosure and is not intending to be limiting. The spatial resolution and signal-to-noise ratio (SNR) of the exemplary system were characterized using imaging experiments on resolution test targets and carbon-fiber phantoms. The biological imaging capabilities of the experimental system were studied using ex ovo chick-embryo chorioallantoic-membrane (CAM) vasculature and imaging melanoma phantoms through a piece of mouse skin.

### Transducer-Fabrication Processes

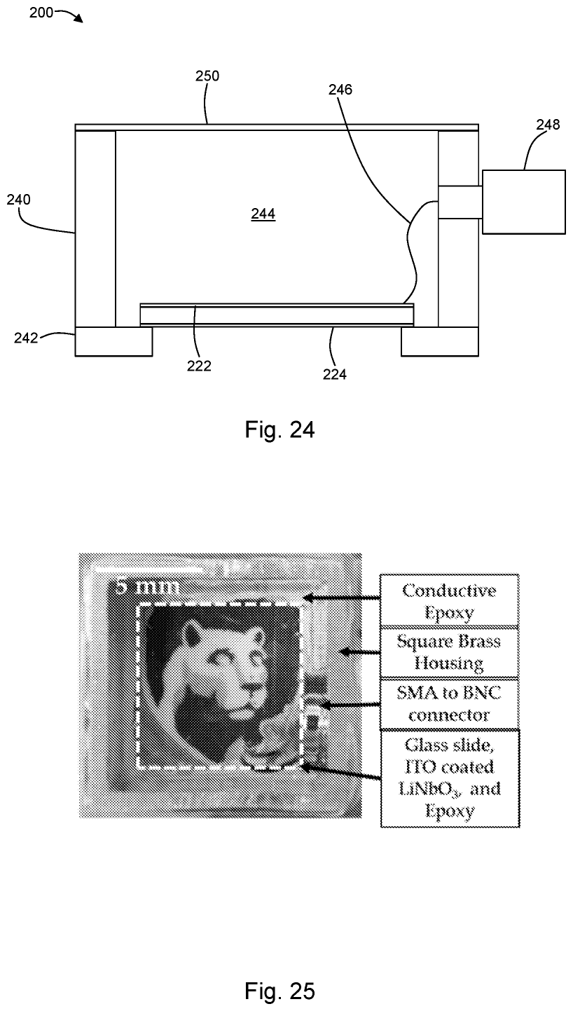

A cross-sectional schematic view of an TUT device 200 is shown in FIG. 24. FIG. 25 shows a photograph of the TUT on top of a Nittany Lion mascot. A 250 μm thick Y-cut 36° LiNbO₃ wafer 220 was sputtered with 200 nm thick indium tin oxide (ITO) electrodes 222,224 on both sides (front side 222 and back side 224) of the wafer and then diced into square pieces of 10×10 mm². The resulting transducer had ˜80% optical transparency in the visible and near-infrared optical-wavelength regions. Square brass tubing (0.5 in. width, 0.032 in. wall thickness, and 10 mm height) was used as a conductive housing 240. The back-side electrode 224 and housing 240 were connected using a conductive silver epoxy 242 (E-solder 3022, Von Roll Isola Inc., New Haven, Conn., USA) that outlined the four edges of the bottom (back-side) electrode 224. The conductive silver epoxy had a thickness of 1 mm and a total width of 2 mm, with 1 mm covering the bottom electrode. This resulted in an field-of-view (FOV) of 9×9 mm². The conductive epoxy also acted as an absorber for surface-acoustic waves generated by the LiNbO₃ in response to pulsed-light incidence.

The top (front-side) electrode 222 was connected to a standard connector (BNC) 248 using a microstranded wire 246. A nonconducting and transparent epoxy (Epotek-301, Epoxy Technologies Inc., Billerica, Mass., USA) 244 was poured until it filled the brass housing 240. This epoxy was used as a backing layer to reduce the ringing effect by absorbing vibrational energy, and improve bandwidth. Extra care was taken to avoid particles being trapped in the epoxy that may have diffracted the light or caused a shadowing effect.

Epoxy is known to shrink during the curing process, which can lead to a curved surface inside the brass housing. This curvature can lead to light diffractions and aberrations. In order to ameliorate this effect, a microglass slide 250 with a thickness of 150 μm was placed on top of the device to form a flat surface.

### Experimental Setup

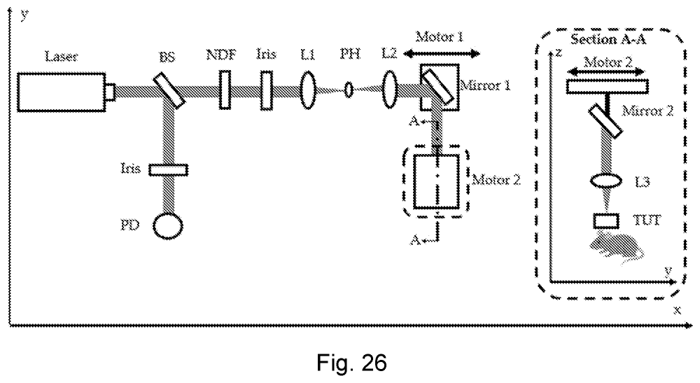

A schematic representing the top-down view of the OR-PAM setup is shown in FIG. 26. The system employed a high-speed pulsed laser (GLPM-10, IPG Photonics; 532 nm wavelength; 1.4 ns pulse duration; tunable pulse-repetition rate in the range of 10-600 kHz; tunable pulse energy between 1.6 and 19 μJ). The 4 mm diameter laser beam passed through a beam sampler (BSF10-A, Thorlabs Inc., Newton, N.J., USA) that diverted 10% of its energy to a photodiode (DET10A, Thorlabs Inc.) used to synchronize with a high-speed (1 gigasample per second) 16 bit data-acquisition system (Razormax-16, Dynamic Signals LLC, Lockport, Ill., USA) connected to a computer.

The remaining 90% of the beam energy passed through a neutral density filter (NDC-50C-4M, Thorlabs Inc., Newton, N.J., USA) and an iris before entering a spatial filter system. A 20 μm pinhole (P20D, Thorlabs Inc., Newton, N.J., USA) and two lenses, with focal lengths 50 mm (LA1131-A, Thorlabs Inc., Newton, N.J., USA) and 75 mm (LA1608, Thorlabs Inc., Newton, N.J., USA) respectively, were then used to filter and collimate the beam. In order to raster-scan the sample for imaging, two motorized stages (NRT-1000, Thorlabs Inc., Newton, N.J., USA) were used to guide the light along the x and y axes for scanning (FIG. 26). A 45° mirror was mounted onto Motor 1, which moved along the x-axis. Motor 2 was mounted on a vertical stage (on the z-axis) and drove another 45° mirror and a 50-mm focal-length planoconvex lens (LA1131-A, Thorlabs Inc., Newton, N.J., USA) along they-axis. The focused light passed through the TUT, which was mounted just above the imaging sample. A thin layer (˜1 mm) of deionized water was used as a coupling medium to receive photoacoustic waves generated from the tissue.

### System and Transducer Characterization

The transducer was evaluated by analyzing its electrical impedance using a vector network analyzer (Agilent E5100A, Keysight Technologies, Inc., Santa Rosa, Calif., USA). Impedance measurements are used to estimate the effective electromechanical coupling coefficient, k_{eff}, of the transducer, which represents its efficiency to convert between electrical and mechanical energy. As seen in FIG. 27a, the resonance and antiresonance frequencies were measured to be 12.05 and 14.22 MHz, respectively, with a resultant k_{eff} of ˜0.53.

Pulse-echo and hydrophone measurements were performed on the TUT using the methods described above. The pulse-echo measurements showed a center frequency of 13 MHz and a fractional bandwidth of 25%, as seen in FIG. 27b. The hydrophone measurements showed a peak pressure of 85 kPa at 5.4 mm distance from the transducer surface. Photoacoustic-pulse response was acquired via a USAF resolution test target (R3L3S1P, Thorlabs Inc., Newton, N.J., USA) and amplified by a preamplifier (Olympus 5073PR, Olympus NDT Inc., Waltham, Mass., USA) with 39 dB gain. The laser energy measured by a pyroelectric energy meter (PE9-ES-C, Ophir-Spiricon, LLC, North Logan, Utah, USA) after spatial filtering was found to be approximately 250 nJ. As seen in FIG. 27c, the received photoacoustic (PA) signal was then averaged 100 times, and showed a center frequency of 13 MHz with a −6 dB fractional bandwidth of 60%. The SNR was calculated as the 20 log₁₀ ratio of the PA signal amplitude to the standard deviation of noise, which was equal to 38 dB.

The lateral resolution of the experimental OR-PAM system was measured by linear scanning along the edge of a ˜2×2 mm² square block of the USAF resolution-test target. During this test, the block was scanned with a 0.5 μm step size. FIG. 27d shows the maximum-amplitude-projection (MAP) image of the square edge, with a dashed line showing the 1 mm long scan length. The experiment data of the edge-spread function are shown in FIG. 27e. The line-spread function was obtained by taking the first derivative of the fitted edge-spread function; its full-width half-maximum (FWHM) showed a lateral resolution of 8.5 μm. The axial resolution of the system was estimated by taking the FWHM of a Gaussian envelope applied to a photoacoustic signal from the target. The FWHM was found to be 0.1 μs, which was equal to 150 μm in water, as seen in FIG. 27f.

The axial resolution of the PAM system is inversely proportional to the bandwidth of the acoustic receiver and was estimated to be 0.88 c/B, where B is the −6 dB bandwidth in MHz and c is the ultrasound velocity inside the tissue medium. Using this relation, the axial resolution of the system was expected to be ˜167 μm, which aligned well with the experimentally observed value of 150 μm. This axial resolution could be further improved by increasing the TUT's bandwidth using a stronger acoustic absorption material as the backing layer. This would also improve the SNR and spatial resolution of the OR-PAM system. Additionally, since no matching layer was used in the experimental TUT, the transmitted acoustic energy propagated through the tissue was ˜17%, considering the acoustic impedances of the LiNbO3 wafer and the tissue were 34 and 1.5 MRayls, respectively. If a transparent matching layer with proper acoustic impedance was added, such as a two-matching-layer design using glass slide and parylene coating, a transmission coefficient as high as ˜45% could be achieved, because the acoustic impedance mismatch between the piezoelectric material and the tissue would be reduced. This would increase the acoustic transmission and receiving sensitivities, and result in an improved SNR.

### Phantom- and Biological-Tissue-Imaging Experiments

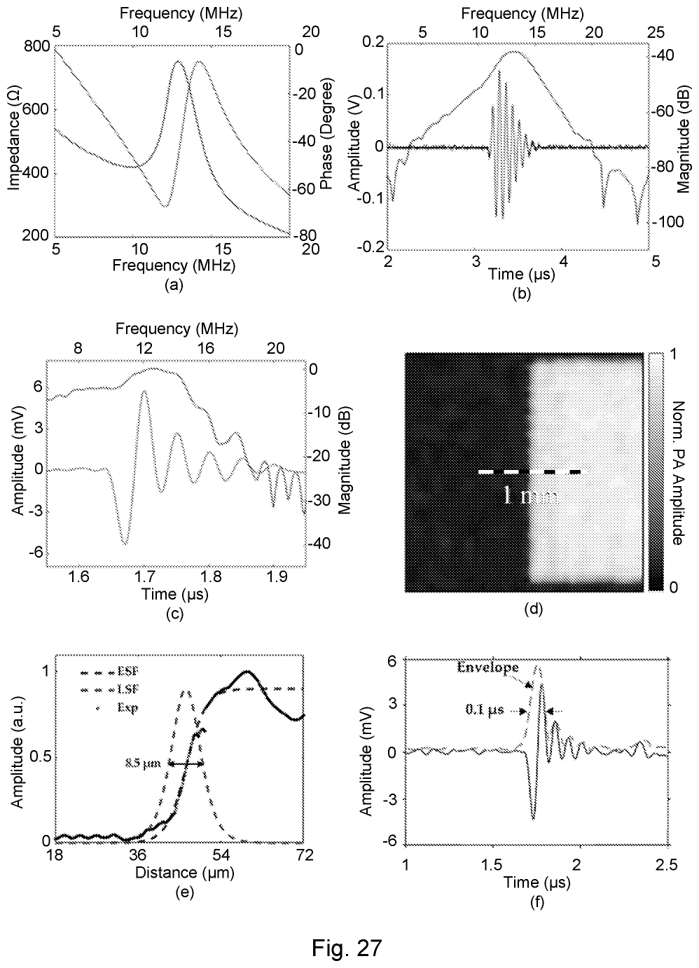

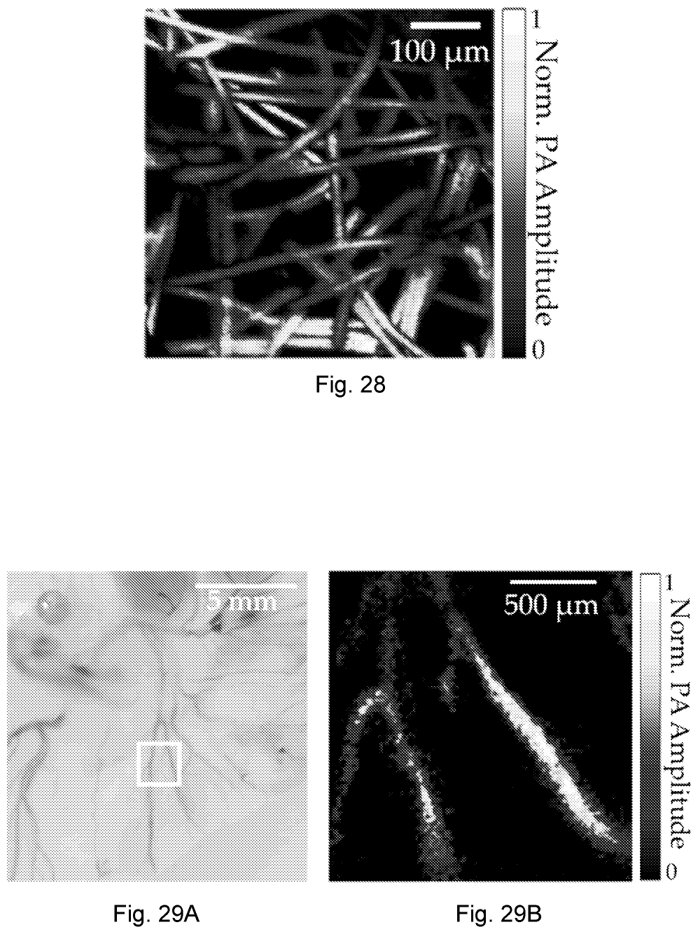

The experimental OR-PAM system was validated by imaging a 12 μm diameter dense carbon-fiber network (that simulated capillary blood vessels) embedded in an agarose phantom gel. Step size was set at 2 μm to cover an area of 0.5×0.5 mm². The photoacoustic signal was then acquired via a high-speed data-acquisition system and averaged 100 times to generate the image. The resulting MAP image of the carbon fiber can be seen in FIG. 28, where each fiber is clearly distinguishable (with sufficient resolution and contrast to be distinguished).

The feasibility of utilizing the OR-PAM for vasculature imaging was demonstrated using chicken embryos. Chicken embryos were used as an animal model to visualize different development phases, and OR-PAM could reveal their important vasculature information for clinical relevance.

For this study, Day 4 fertile chicken eggs (E4) were obtained from the Poultry Education and Research Center (PERC) at The Pennsylvania State University. The eggs were gently cracked, and the embryos carefully placed on weigh boats under sterile conditions. The embryos were then incubated at 38° C. with 3% CO₂ in a humidified incubator. For imaging, the CAM attached to each embryo was removed by cutting around its edges, and then each embryo was quickly and gently transferred to a Petri dish and rinsed with deionized water. Finally, each embryo was placed on top of an agarose-gel phantom bed for imaging. FIG. 29A shows a photograph of the CAM: the scanning area is marked by a white box. The scan step size was set at 20 μm to cover a 2×2 mm² area, and imaging data were averaged 500 times to improve SNR. The vasculature image from MAP, seen in FIG. 29B, clearly shows the vascular-branch pattern marked in FIG. 29A.

The experimental OR-PAM's application in melanoma imaging was demonstrated by scanning a melanoma phantom. The depth of melanoma invasion under the skin, also known as Breslow's depth, is one of the three most important prognostic factors in melanoma detection, and it reveals important details about how tumor cells invade. To demonstrate the feasibility of TUT-based OR-PAM wearable imaging of melanoma patients without the need for thick gel coupling, the following melanoma-tissue experiment was conducted.

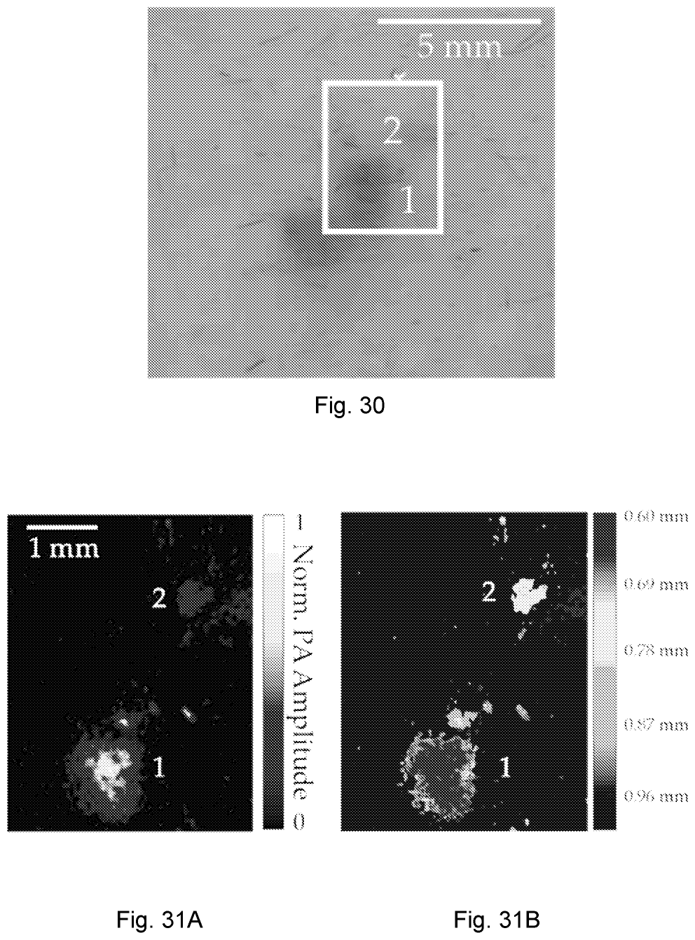

Approximately 2 mg of melanin particles (M8631, Sigma Aldrich, St. Louis, Mo., USA; optical absorption coefficient ˜1100 cm⁻¹ at 532 nm) was mixed with 100 mg of 1.5% agarose phantom gel and placed under a piece of mouse skin at different depths. The scan area was set as 3.5×4.5 mm² to cover two melanoma spots under the skin, as shown by the white box region in FIG. 30.

A pulse energy after the spatial filter was set at ˜600 nJ to yield an optical fluence of ˜14.7 mJ/cm², which was below the American National Standards Institute (ANSI) safety skin maximum permissible exposure (MPE) limit of 20 mJ/cm² at 532 nm. A-lines were averaged 300 times to generate the MAP image shown in FIG. 31A. The image was then color-coded based on distance relative to the skin surface, which showed clear melanoma boundaries and depth information (FIG. 31B). The feasibility of the high-contrast melanoma imaging demonstrated here could benefit clinical point-of-care depth detection and the monitoring of melanoma cells using wearable TUT-based OR-PAM. During the scanning, blood diffusion from certain areas of the ex ovo samples resulted in low-contrast vascular images: vascular contrast is expected to improve when imaging living subjects in vivo.

Scan speeds of up to 1000×1000 steps (or more) in 100 s may be achievable using scanning methods such as galvo-mirror-based scanning of the optical beam like that employed in conventional OR-PAM systems.

Although the present disclosure has been described with respect to one or more particular embodiments, it will be understood that other embodiments of the present disclosure may be made without departing from the spirit and scope of the present disclosure.

What is claimed is:

1. A device for photoacoustic imaging, comprising:
a transparent piezoelectric transducer for receiving ultrasonic emission from a region of interest;
a light source configured to illuminate the region of interest, wherein the illumination is provided at least partially through the transducer.

2. The device of claim 1, wherein the transducer comprises a piezoelectric substrate made from lithium niobate (LiNbO₃), polyvinylidene fluoride (PVDF), lead magnesium niobate-lead titanate (PMN-PT), a piezoelectric composite, or combinations thereof.

3. The device of claim 2, further comprising a front electrode in communication with a front side of the substrate.

4. The device of claim 3, wherein the front electrode is a layer of a transparent conductor.

5. The device of claim 4, wherein the transparent conductor is Indium-Tin-Oxide (ITO).

6. The device of claim 3, further comprising a back electrode in communication with a back side of the transducer.

7. The device of claim 1, wherein the light source has a source wavelength range and the transducer is transparent in the source wavelength range.

8. The device of claim 7, wherein the transducer is configured to transmit at least 30% of light in the source wavelength range.

9. The device of claim 7, wherein the source wavelength range is 250 nm-2400 nm.

10. The device of claim 1, wherein the light source comprises a laser and/or a light-emitting diode.

11. The device of claim 1, wherein the light source comprises one or more optical fibers.

12. The device of claim 11, wherein the optical fiber is configured to be coupled to a laser.

13. The device of claim 1, further comprising a transparent backing layer on a back side of the transducer.

14. The device of claim 1, wherein the transducer comprises more than one piezoelectric elements arranged in a linear array.

15. The device of claim 1, wherein the transducer comprises more than one piezoelectric elements arranged in a planar array.

16. The device of claim 1, further comprising a housing connected to the transducer.

17. An endoscope comprising a device according to claim 1.

18. A method of photoacoustic imaging a region of interest, comprising:
providing a transparent piezoelectric transducer;
illuminating a first portion of the region of interest through the transducer; and
receiving an ultrasonic emission from the first portion of the region of interest, wherein the ultrasonic emission results from the illumination.

19. The method of claim 18, further comprising:
moving the transducer to a second portion of the region of interest;
illuminating the second portion of the region of interest through the transducer; and
receiving an ultrasonic emission from the second portion of the region of interest, wherein the ultrasonic emission results from the illumination.

20. The method of claim 19, wherein the steps are repeated for additional portions of the region of interest.

21. The method of claim 18, further comprising:
illuminating a second portion of the region of interest through the transducer; and
receiving an ultrasonic emission from the second portion of the region of interest, wherein the ultrasonic emission results from the illumination.

22. The method of claim 18, further comprising actuating the transducer to excite at least a portion of the region of interest.

23. The method of claim 22, further comprising receiving an ultrasonic emission from the at least a portion of the region of interest, wherein the ultrasonic emission results from the excitation by the transducer.

24. The method of claim 22, further comprising monitoring the region of interest using a detector.
